(19) United States
(12) Patent Application Publication
(10) Pub. No.: US 2024/0136050 A1
(43) Pub. Date: Apr. 25, 2024

(54) CUSTOMIZABLE NEUROMODULATION DEVICE TREATMENT JOURNEY

(71) Applicant: Boston Scientific Neuromodulation Corporation, Valencia, CA (US)

(72) Inventors: Kyle Harish Srivastava, Saint Paul, MN (US); Bradley Lawrence Hershey, Carrollton, TX (US); Amarpreet Singh Bains, Woodbury, MN (US)

(21) Appl. No.: 18/380,513

(22) Filed: Oct. 15, 2023

Related U.S. Application Data
(60) Provisional application No. 63/417,815, filed on Oct. 20, 2022.

(51) Int. Cl. G16H 20/70; G16H 40/60

(52) U.S. Cl. CPC G16H 20/70; G16H 40/60

(57) ABSTRACT

Systems, devices, and methods for providing customizable interactive healthcare services in relation to neuromodulation treatment are disclosed. A digital health system includes a memory device that stores a database of device operation and treatment (DOT) information of neuromodulation device. The DOT information includes information releasable to a patient and device programming capability authorized to the patient, and can be categorized according to a plurality of pre-determined treatment phases and/or according to a plurality of pre-determined patient skill or engagement levels with the neuromodulation device. A processor determines a treatment phase and a skill or engagement level using patient state information, queries the database to determine personalized DOT information corresponding to the treatment phase and the patient skill or engagement level, and provides the personalized DOT information to the patient. The authorized programming capability allows the patient to adjust the neurostimulation therapy.

### CLAIM OF PRIORITY

This application claims the benefit of U.S. Provisional Application No. 63/417,815, filed on Oct. 20, 2022, which is hereby incorporated by reference in its entirety.

### TECHNICAL FIELD

The present disclosure relates generally to medical devices, and more particularly, to systems, devices, and methods for providing customizable healthcare services in relation to neuromodulation device treatment in a patient.

### BACKGROUND

Chronic pain, such as pain present most of the time for a period of six months or longer during the prior year, is a highly pervasive complaint and consistently associated with psychological illness. Chronic pain may originate with a trauma, injury or infection, or there may be an ongoing cause of pain. Chronic pain may also present in the absence of any past injury or evidence of body damage. Common chronic pain can include headache, low back pain, cancer pain, arthritis pain, neurogenic pain (pain resulting from damage to the peripheral nerves or to the central nervous system), or psychogenic pain (pain not due to past disease or injury or any visible sign of damage inside or outside the nervous system

Neurostimulation, also referred to as neuromodulation, has been proposed as a therapy for a number of conditions including chronic pain. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system can include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system.

A neurostimulation system has been used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to produce an analgesic effect that masks pain sensation, or to produce a functional effect that allows increased movement or activity of the patient. Other forms of neurostimulation may include a DBS system which uses similar pulses of electricity at particular locations in the brain to reduce symptoms of essential tremors, Parkinson's disease, psychological disorders, or the like.

The capability of a neurostimulation system largely depends on its programmability. Neurostimulation systems are typically programmed by a clinician or a system expert in a clinical setting using a clinical programmer. For example, a clinician may use the clinical programmer to set one or more stimulation parameters (e.g., pulse voltage or current amplitude, pulse width, or pulse rate), select an electrostimulation program (as defined by a group of parameter values) for an electrical stimulation therapy to be delivered to the patient, or program active electrodes to deliver the electrostimulation pulses.

Some neurostimulation systems include a patient programmer that enables a patient to interact with his or her neuromodulation device (also referred to as “neurostimulation device”), such as activating or deactivating a neurostimulation therapy, changing a stimulation parameter value, or switching between stimulation programs, among other adjustments of device settings. The patient programmer allows the patients to directly tune the neurostimulation therapy to meet personal needs in an ambulatory setting without frequent clinic visits. For example, a patient receiving SCS therapy for pain management may experience gradual physiological, functional, or emotional changes, which can get unnoticed for an extended period of time when the patient is outside a clinical setting. A patient programmer allows the patient to identify such changes and adjust the SCS therapy accordingly. Adequate information about disease progression, utilities and functions of the neuromodulation devices, and treatment options and therapy programming guidance are desirable to help the patient make better use of their devices and improve treatment outcome.

### SUMMARY

This document discusses systems, devices, and methods for providing customizable neuromodulation device and treatment information to a patient to assist in device programming. According to one example, a digital health system includes a memory device that stores and maintains a database of device operation and treatment (DOT) information with respect to a neuromodulation device, such as an implantable neurostimulator configured to provide spinal cord stimulation (SCS) therapy for pain management. The database includes information releasable to a patient with respect to the disease or medical conditions of the patient, utilities and functionalities of the neuromodulation device, neuromodulation therapy options, device operation instructions and programming guidance, and trackable treatment goals, etc. The DOT information may additionally or alternatively include device programming capability authorized to the patient. The DOT information in the database can be categorized according to a plurality of pre-determined treatment phases and/or according to a plurality of pre-determined patient skills or engagement levels with the neuromodulation device. A processor of the system can determine a treatment phase in the treatment journey and the patient's skill or engagement level at that treatment phase using patient state information. The processor can query the database to determine personalized DOT information corresponding to the treatment phase and the patient's skill or engagement level, and interactively provide the personalized DOT information to the patient on a user interface in accordance with a personalized mode of interaction with the patient that can also be determined based on the patient's skill or engagement level and the treatment phase.

Example 1 is a system for providing customizable interactive healthcare services to a patient in relation to neuromodulation device treatment. The system comprises: a memory device configured to store and maintain a database of device operation and treatment (DOT) information of a neuromodulation device, the database of DOT information being categorized according to a plurality of pre-determined treatment phases and a plurality of pre-determined patient skills or engagement levels with the neuromodulation device; and a processor configured to: determine a treatment phase and a skill or engagement level of the patient at the determined treatment phase using patient state information; query the database to identify therefrom personalized DOT information that corresponds to the determined treatment phase and the skill or engagement level of the patient; and provide the personalized DOT information to the patient on a user interface.

In Example 2, the subject matter of Example 1 optionally includes, wherein the processor is further configured to determine a personalized mode of interaction with the patient based on the determined treatment phase and the skill or engagement level of the patient, and to provide the personalized DOT information to the patient in accordance with the personalized mode of interaction.

In Example 3, the subject matter of any one or more of Examples 1-2 optionally include, wherein the patient state information includes text or voice input from the patient via the user-interface device, wherein the processor is configured to process the text or voice input using natural language processing, and to determine the treatment phase and the skill or engagement level of the patient using the processed text or voice input.

In Example 4, the subject matter of any one or more of Examples 1-3 optionally include, wherein the processor is configured to: detect a change in treatment phase or a change in patient skills or engagement level; query the database to update the personalized DOT information; and provide the updated personalized DOT information to the patient on the user interface.

In Example 5, the subject matter of any one or more of Examples 1-4 optionally include, wherein the plurality of pre-determined treatment phases include a plurality of pre-determined temporal phases representing respective times elapsed from a medical event or a predefined milestone.

In Example 6, the subject matter of any one or more of Examples 1-5 optionally include, wherein the plurality of pre-determined treatment phases include a plurality of pre-determined stages of patient experience with a neuromodulation therapy.

In Example 7, the subject matter of any one or more of Examples 1-6 optionally include, wherein the plurality of pre-determined patient skills or engagement levels include a novice level, an intermediate level, and an expert level, wherein the processor is configured to determine the skill or engagement level of the patient as one of the novice level, the intermediate level, or the expert level using the patient state information.

In Example 8, the subject matter of any one or more of Examples 1-7 optionally include, wherein the plurality of pre-determined patient skills or engagement levels include a plurality of pre-determined patterns of patient interaction with the neuromodulation device to adjust a neuromodulation therapy parameter, wherein the processor is configured to determine a device interaction pattern of the patient using the patient state information, and query the database to identify therefrom the personalized DOT information that corresponds to the determined treatment phase and the determined device interaction pattern.

In Example 9, the subject matter of any one or more of Examples 1-8 optionally include, wherein the patient state information includes patient past interactions with the neuromodulation device, wherein the processor is configured to track the patient past interactions with the neuromodulation device over time, and to determine the skill or engagement level of the patient based on the tracked patient past interactions.

In Example 10, the subject matter of any one or more of Examples 1-9 optionally include, wherein the processor is configured to initiate a diagnostic test of patient performance in interacting with the neuromodulation device, and to determine the skill or engagement level of the patient based on the patient performance in the diagnostic test.

In Example 11, the subject matter of any one or more of Examples 1-10 optionally include, wherein the DOT information in a first category of pre-determined treatment phase or pre-determined patient skill or engagement level is different from the DOT information in a second category of pre-determined treatment phase or pre-determined patient skill or engagement level.

In Example 12, the subject matter of any one or more of Examples 1-11 optionally include, wherein the database of DOT information includes categorized device and therapy information releasable to the patient, including information about one or more of: device functions; device operation instructions; neuromodulation therapy programs; or trackable neuromodulation treatment goals.

In Example 13, the subject matter of Example 12 optionally includes, wherein the pre-determined patient skills or engagement levels include a first skill or engagement level and a second skill or engagement level, the second skill or engagement level representing a lower level of skill or engagement with the neuromodulation device than the first skill or engagement level, wherein the DOT information corresponding to the first skill or engagement level includes a higher volume or more advanced device and therapy information releasable to the patient than the DOT information corresponding to the second skill or engagement level.

In Example 14, the subject matter of any one or more of Examples 1-13 optionally include, wherein the database of DOT information includes categorized device programming capability authorized to the patient, wherein the processor is configured to, upon a user request, enable the authorized device programming capability corresponding to the determined treatment phase and the identified patient skills or engagement level.

In Example 15, the subject matter of Example 14 optionally includes, wherein the pre-determined patient skills or engagement levels include a first skill or engagement level and a second skill or engagement level, the second skill or engagement level representing a lower level of skill or engagement with the neuromodulation device than the first skill or engagement level, wherein the authorized device programming capability corresponding to the first skill or engagement level includes more advanced device programming capability authorized to the patient than the device operation and treatment programming capability corresponding to the second skill or engagement level.

Example 16 is a method of providing customizable interactive healthcare services to a patient in relation to neuromodulation device treatment. The method comprises steps of: providing a database of device operation and treatment (DOT) information of a neuromodulation device, the database of DOT information being categorized according to a plurality of pre-determined treatment phases and a plurality of pre-determined patient skills or engagement levels with the neuromodulation device; determining a treatment phase and a skill or engagement level of the patient at the determined treatment phase using patient state information; querying the database to identify therefrom personalized DOT information that corresponds to the determined treatment phase and the skill or engagement level of the patient; and providing the personalized DOT information to the patient on a user interface.

In Example 17, the subject matter of Example 16 optionally includes: determining a personalized mode of interaction with the patient based on the determined treatment phase and the skill or engagement level of the patient; and providing the personalized DOT information in accordance with the personalized mode of interaction.

In Example 18, the subject matter of any one or more of Examples 16-17 optionally include, wherein the patient state information includes text or voice input from the patient via the user-interface device, wherein determining the treatment phase and the skill or engagement level of the patient includes processing the text or voice input using natural language processing, and determining the treatment phase and the skill or engagement level of the patient using the processed text or voice input.

In Example 19, the subject matter of any one or more of Examples 16-18 optionally include, wherein the plurality of pre-determined treatment phases include a plurality of pre-determined temporal phases representing respective times elapsed from a medical event or a predefined milestone, or plurality of pre-determined stages of patient experience with a neuromodulation therapy.

In Example 20, the subject matter of any one or more of Examples 16-19 optionally include, wherein the DOT information in a first category of pre-determined treatment phase or pre-determined patient skill or engagement level is different from the DOT information in a second category of pre-determined treatment phase or pre-determined patient skill or engagement level.

In Example 21, the subject matter of any one or more of Examples 16-20 optionally include, wherein the database of DOT information includes categorized device and therapy information releasable to the patient, wherein the pre-determined patient skills or engagement levels include a first skill or engagement level and a second skill or engagement level, the second skill or engagement level representing a lower level of skill or engagement with the neuromodulation device than the first skill or engagement level, wherein the DOT information corresponding to the first skill or engagement level includes a higher volume or more advanced device and therapy information releasable to the patient than the DOT information corresponding to the second skill or engagement level.

In Example 22, the subject matter of any one or more of Examples 16-21 optionally include, wherein the database of DOT information includes categorized device programming capability authorized to the patient, wherein the pre-determined patient skills or engagement levels include a first skill or engagement level and a second skill or engagement level, the second skill or engagement level representing a lower level of skill or engagement with the neuromodulation device than the first skill or engagement level, wherein the authorized device programming capability corresponding to the first skill or engagement level includes more advanced device programming capability authorized to the patient than the device operation and treatment programming capability corresponding to the second skill or engagement level.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an neurostimulation system.

FIG. 2 illustrates, by way of example and not limitation, a stimulation device and a lead system, such as may be implemented in the neurostimulation system of FIG. 1.

FIG. 3 illustrates, by way of example and not limitation, a programming device, such as may be implemented in the neurostimulation system of FIG. 1.

FIG. 4 illustrates, by way of example and not limitation, an implantable neurostimulation system and portions of an environment in which the system may be used.

FIG. 5 illustrates, by way of example and not limitation, an implantable stimulator and one or more leads of a neurostimulation system, such as the implantable neurostimulation system of FIG. 4.

FIG. 6 illustrates, by way of example and not limitation, a programming system and data analysis system for use with a neurostimulation system, such as the implantable neurostimulation system of FIG. 4.

FIG. 7 illustrates, by way of example and not limitation, an assisted therapy programming system for providing customizable interactive healthcare services in relation to neuromodulation treatment via a neuromodulation device.

FIGS. 8A-8B illustrate, by way of example, DOT information database represented by a two-dimensional data array.

FIGS. 9A-9B illustrate, by way of example and not limitation, user interfaces on a patient programmer that provides customized interactive presentation of DOT information in accordance with the skill or engagement level of the patient and the treatment phase.

FIG. 10 illustrates, by way of example and not limitation, a method of providing customizable interactive healthcare services in relation to neuromodulation device treatment in a patient.

FIG. 11 is a block diagram illustrating generally an example machine upon which any one or more of the techniques (e.g., methodologies) discussed herein may perform.

### DETAILED DESCRIPTION

A patient programmer in a neuromodulation device system provides an information exchange and device control platform that enables the patient to interact with his or her neuromodulation device and make therapy adjustments. Although a convenient tool, conventional patient programmers have some limitations that may prevent the patients from maximizing the benefit of the neuromodulation therapies. One of such limitations is the “one-size-fits-all” type of information presentation and device programmability, where the device utility and therapy information and/or the device programming capability are not customizable to meet individual patient's need, but are universally provided to all patients regardless of inter-patient variabilities in cognitive performances; knowledge, experience, and skills with neuromodulation devices; or general inclination or engagement levels with device therapies. For patients with little knowledge and experience with neuromodulation devices and the variety of neuromodulation therapies, universal information and universal programming capabilities can be too difficult to comprehend, or too excessive to follow. The patients may feel frustrated by not knowing what to expect during their treatment journey or when or how to adjust stimulation parameters. On the other hand, for more experienced and knowledgeable patients, or patients more engaged with neuromodulation therapy programs, the universal device utility and therapy information and the universal programming capabilities can be inadequate to meet their needs to explore different treatment options to find an optimal treatment program. Additionally, the “one-size-fits-all” scheme also lacks flexibility to accommodate a patient's changing health status and conditions. For example, patients may experience new or evolving medical events, or encounter device issues during the treatment journey. Their skills and engagement levels with the neuromodulation device may change over time. Universal device utility and therapy information and universal programming capabilities may not meet patient's changing needs along the treatment journey.

The present inventors have recognized an unmet need to improve the functionalities of patient programmers to provide customized information to the patient with respect to utilities and functionalities of neuromodulation devices, treatment options, and expectations of the therapies at different stages of neuromodulation treatment. Disclosed herein are various embodiments of systems and techniques that enable customizable interactive healthcare services to a patient in relation to neuromodulation device treatment, including customizable device and treatment information releasable to the patient and device programming capabilities authorized toe the patient that assist the patient in programming the neuromodulation device. According to one example, a digital health system can include a memory device to store and maintain a database of device operation and treatment (DOT) information categorized according to a plurality of pre-determined treatment phases and/or according to a plurality of pre-determined patient skill or engagement levels with the neuromodulation device. The categorized DOT information includes information releasable to a patient and device programming capability authorized to the patient. A processor can determine a treatment phase of the patient's treatment journey and the patient's skill or engagement level at that treatment phase using patient state information, query the database to determine personalized DOT information that corresponds to the treatment phase and the skill or engagement level of the patient, and interactively provide the personalized DOT information to the patient on a user interface. The DOT information may be presented in accordance with a personalized mode that can be determined based on the treatment phase and the skill or engagement level of the patient.

Various embodiments of the customizable interactive presentation of information and therapy programming capabilities as described in this document can provide individualized information, guidance, and assistance to patients to make better use of their neuromodulation devices. The customizable interactive information presentation and therapy programming offerings can be provided at each of a plurality of phases of treatment journey. In some examples, an in-app timeline feature can be created and displayed to the patient to keep the patient informed about the present phase of treatment, therapy options available and expected outcomes at present and future treatment phases. Such a feature in a patient programmer can mitigate the general lack of communication from the clinician to the patient with respect to treatment and patient expectations therefrom following an implant of permanent neuromodulation device. The customizable interactive information presentation and therapy programming capabilities can provide the patient with an enhanced user experience, promote patient engagement with their devices and therapies for a longer time in order to achieve better treatment outcome (e.g., SCS therapy for pain management), and encourage active and informed exploration of various therapy programs. Accordingly, with the customizable interactive information presentation and therapy programming offerings in accordance with various embodiments as described in this document, higher acceptance rates of neuromodulation therapy, more conversions from a device trial to permanent implants of neuromodulation devices, more active engagement with acceptance of novel therapy programs, and fewer explants of implantable neuromodulation can be achieved. Additionally, by adapting information presentation and device programming capabilities to patient personal skills or engagement levels and phases of a treatment journey, the amount and/or the type of device and therapy information can vary over time and provided to the patient based on patient's need or personal preference, such that the usages of device resources (e.g., power, memory, computation and data processing) can be optimized, and the overall utility and functionality of the neuromodulation devices can be improved.

In various examples, the present document describes a platform in a medical device system that facilitates patients' interaction with their neuromodulation devices to receive customized device and treatment information and guidance on device operation and therapy programming. The information can be presented, and the patient input or feedback can be collected, in various forms of freeform text, voice, or other unstructured or unclassified data formats. In some examples, the patients may use the platform (e.g., an app in a smartphone or other mobile devices) to set their treatment goals and track the progress towards the goal. The freeform text or voice may be processed using text or voice analysis methods, such as natural language processing (NLP). Information from the evaluation of freeform text may be used to provide informational content to a patient or to a clinician (e.g., to present guidance regarding the effects of treatment or ways to improve treatment outcomes). In addition to use with aspects of programs and programming values, information from the evaluation of freeform text may also be used to cause device actions (e.g., to run diagnostics on the neuromodulation device). In still other examples, information from the evaluation of freeform text may also be used to provide a clinical triage system, or to update data records, among other effects.

In various embodiments, the present subject matter may be implemented using a combination of hardware and software designed to capture and analyze freeform text, voice, or other unstructured information from users, and related device data or context from a neuromodulation treatment. For instance, some examples are provided with reference to a mobile computing device (e.g., smartphone) app executing a user interface to collect freeform text, entered in the form of questions or voice commands. Other examples are provided with reference to a computing system implemented via a chatbot (e.g., generating data for a smartphone app chat session or SMS message chat session) that presents questions or replies, in an effort to collect and process patient input provided in text (e.g., provided directly in freeform text from a patient response, provided from converted voice-to-text responses, or provided directly or indirectly with other interactions with various parties or entities). Still other examples are provided with reference to a computing system platform which captures and evaluates data from sensors (e.g., wearable devices, implantable devices, or the neuromodulation device) that can be used to cross-reference or correlate freeform text statements from a patient. Many of the following approaches are provided with specific reference to text analysis and NLP, but it will be understood that such approaches may be supplemented or substituted with other technical implementations of text processing and data analysis involving including artificial intelligence (AI), including models implementing machine learning, neural networks, decision trees, and the like. Such freeform text processing capabilities, when incorporated into the customizable interactive information presentation and therapy programming platform, allows for more convenient and efficient therapy adjustment, and advantageously promotes patient engagement with their neuromodulation devices and active exploration of device functions or advanced therapy options.

It will be understood that a variety of the following embodiments may be operated to provide users such as patients, caregivers, clinicians, researchers, physicians, or others with the ability to monitor, collect and provide feedback, and adapt neurostimulation programs and neurostimulation effects (including, neurostimulation programming that provides a variation in the location, intensity, and type of defined waveforms and patterns in an effort to increase therapeutic efficacy and/or patient satisfaction). While neurostimulation therapies, such as SCS therapy, are specifically discussed as examples, the present subject matter may apply to other therapies that employs stimulation pulses of electrical or other forms of energy for treating chronic pain or like physiological or psychological conditions.

The delivery of neurostimulation energy that is discussed herein may be delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Many current neurostimulation systems are programmed to deliver periodic pulses with one or a few uniform waveforms continuously or in bursts. However, neural signals may include more sophisticated patterns to communicate various types of information, including sensations of pain, pressure, temperature, etc. Accordingly, the following drawings provide an introduction to the features of an example neurostimulation system and how such programming may be accomplished through open-loop or closed-loop neurostimulation systems, and integrated with the present data analysis platforms.

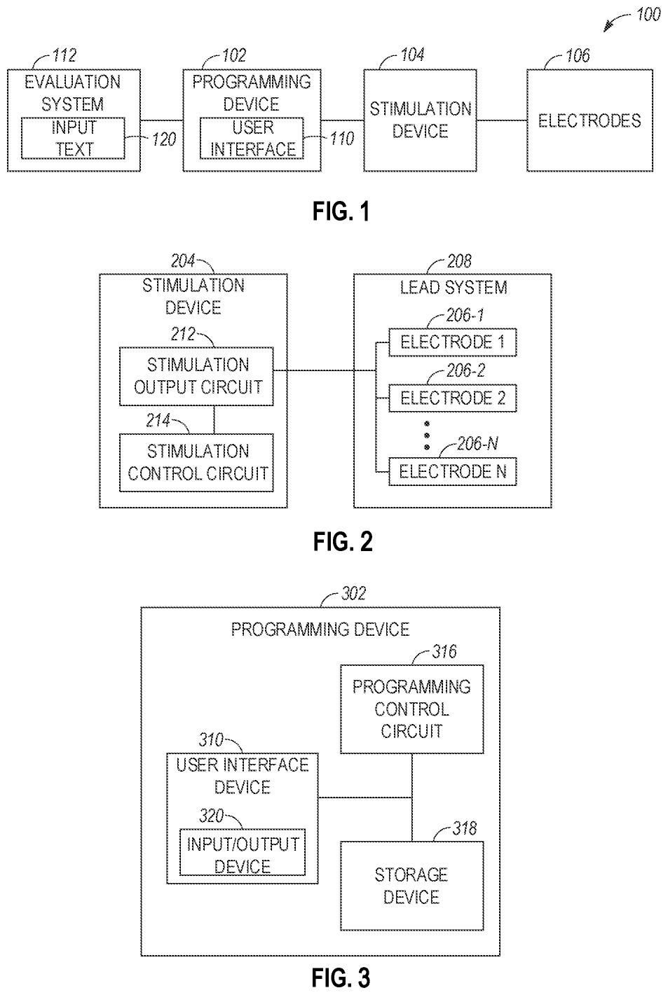

FIG. 1 illustrates an embodiment of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are selected or programmable by a clinical user, such as a physician or other caregiver who treats the patient using system 100; however, some of the parameters may also be provided in connection with closed-loop programming logic and adjustment. Programming device 102 provides the user with accessibility to implement, change, or modify the programmable parameters. In various embodiments, programming device 102 is configured to be communicatively coupled to stimulation device 104 via a wired or wireless link.

In various embodiments, programming device 102 includes a user interface 110 (e.g., a user interface embodied by a graphical, text, voice, or hardware-based user interface) that allows the user to set and/or adjust values of the user-programmable parameters by creating, editing, loading, and removing programs that include parameter combinations such as patterns and waveforms. These adjustments may also include changing and editing values for the user-programmable parameters or sets of the user-programmable parameters individually (including values set in response to a therapy efficacy indication). Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

The present approaches further provide examples of an evaluation system 112, such as a data analysis system, which is used to adapt, modify, start, stop, monitor, or identify a neuromodulation treatment with stimulation device 104. The evaluation system 112 can be associated with, or included into, the programming device 102. The evaluation system 112 initiates an action related to the neuromodulation treatment based on text analysis performed on input text 120. The input text 120 can be in forms of freeform text, voice, or other unstructured or unclassified data formats from users. The input text 120 may be directly collected from the patient or other system users (such as via the user interface 110 of the programming device 102) and analyzed by the evaluation system 112, to then cause a programming effect in the programming device 102, and the stimulation device 104, and the neuromodulation treatment provided by the electrodes 106. The user input may be used to select, load, modify, implement, measure, analyze, or evaluate one or more parameters of a defined program for neuromodulation treatment that is implemented by the stimulation device 104, or the operation of the stimulation device 104. In some examples, the user input may contain information about patient feedback on treatment (e.g., a therapy program provided by the neuromodulation device). The patient input or feedback may be evaluated using one or more analytical methods including, for example, natural language processing (NLP), sentiment analysis, rules, and other operational or treatment objectives that are identified. Various logic or algorithms can then determine an appropriate action to take based on the state of the patient, including but not limited to: a program or parameter change or recommendation to produce an improvement for a treatment objective (such as to address pain, increase mobility, reduce sleep disruption, and the like); diagnostic or remedial actions on the stimulation device 104; data logging or alerts to the patient or a clinician associated with the patient; and the like.

Example parameters that can be implemented by a selected neurostimulation program include, but are not limited to the following: amplitude, pulse width, frequency, duration, total charge injected per unit time, cycling (e.g., on/off time), pulse shape, number of phases, phase order, interphase time, charge balance, ramping, as well as spatial variance (e.g., electrode configuration changes over time). As detailed in FIG. 6, a controller, e.g., controller 630 of FIG. 6, can implement program(s) and parameter setting(s) to affect a specific neurostimulation waveform, pattern, or energy output, using a program or setting in storage, e.g., external storage device 616 of FIG. 6, or using settings communicated via an external communication device 618 of FIG. 6 corresponding to the selected program. The implementation of such program(s) or setting(s) may further define a therapy strength and treatment type corresponding to a specific pulse group, or a specific group of pulse groups, based on the specific programs or settings. The evaluation system 112 and the evaluation of the input text 120 provide a mechanism to determine the effectiveness of such programs or settings, and to identify issues and provide remediation for ineffective programs or settings, offer suggestions or recommendations for new programs or settings, or even to automatically change programs or settings.

In some examples, the input text 120 received from the patient or other system users can include information about patient inquiries about device functions or operations, treatment operations (e.g., electrostimulation programs) available for them to use, expectations of treatment outcome, patient personalized treatment objectives or intended usage of the neuromodulation device, patient feedback on treatment, etc. The patient input or feedback can be provided in response to or interactively with system-generated customized presentation of information about neuromodulation device and treatment and customized therapy programming capabilities authorized to the patient. Such presentation of information and therapy programming capabilities are customized in accordance with patient skills or engagement levels with their neuromodulation devices and/or the phases of treatment journey. In some examples, the input text 120 may contain information about a personalized treatment goal of the patient. The stimulation device 104 to deliver the neuromodulation treatment to the patient via the electrodes 106 to help the patient achieve the treatment goal.

Portions of the evaluation system 112, the stimulation device 104 (e.g., implantable medical device), or the programming device 102 can be implemented using hardware, software, or any combination of hardware and software. Portions of the stimulation device 104 or the programming device 102 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. The system 100 could also include a subcutaneous medical device (e.g., subcutaneous ICD, subcutaneous diagnostic device), wearable medical devices (e.g., patch-based sensing device), or other external medical devices.

FIG. 2 illustrates an embodiment of a stimulation device 204 and a lead system 208, such as may be implemented in neurostimulation system 100 of FIG. 1. Stimulation device 204 represents an embodiment of stimulation device 104 and includes a stimulation output circuit 212 and a stimulation control circuit 214. Stimulation output circuit 212 produces and delivers neurostimulation pulses, including the neurostimulation waveform and parameter settings implemented via a program selected or implemented with the user interface 110. Stimulation control circuit 214 controls the delivery of the neurostimulation pulses using the plurality of stimulation parameters, which specifies a pattern of the neurostimulation pulses. Lead system 208 includes one or more leads each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes. Those of ordinary skill in the art will understand that the neurostimulation system 100 may include additional components such as sensing circuitry for patient monitoring and/or feedback control of the therapy, telemetry circuitry, and power. The neurostimulation system 100 may also integrate with other sensors, or such other sensors may independently provide information for use with programming of the neurostimulation system 100.

The neurostimulation system may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. Other parameters that may be controlled or varied include the amplitude, pulse width, and rate (or frequency) of the electrical pulses. Each electrode configuration, along with the electrical pulse parameters, can be referred to as a “modulation parameter” set. Each set of modulation parameters, including fractionalized current distribution to the electrodes (as percentage cathodic current, percentage anodic current, or off), may be stored and combined into a program that can then be used to modulate multiple regions within the patient.

The neurostimulation system may be configured to deliver different electrical fields to achieve a temporal summation of modulation. The electrical fields can be generated respectively on a pulse-by-pulse basis. For example, a first electrical field can be generated by the electrodes (using a first current fractionalization) during a first electrical pulse of the pulsed waveform, a second different electrical field can be generated by the electrodes (using a second different current fractionalization) during a second electrical pulse of the pulsed waveform, a third different electrical field can be generated by the electrodes (using a third different current fractionalization) during a third electrical pulse of the pulsed waveform, a fourth different electrical field can be generated by the electrodes (using a fourth different current fractionalized) during a fourth electrical pulse of the pulsed waveform, and so forth. These electrical fields can be rotated or cycled through multiple times under a timing scheme, where each field is implemented using a timing channel. The electrical fields may be generated at a continuous pulse rate, or as bursts of pulses. Furthermore, the interpulse interval (i.e., the time between adjacent pulses), pulse amplitude, and pulse duration during the electrical field cycles may be uniform or may vary within the electrical field cycle. Some examples are configured to determine a modulation parameter set to create a field shape to provide a broad and uniform modulation field such as may be useful to prime targeted neural tissue with sub-perception modulation. Some examples are configured to determine a modulation parameter set to create a field shape to reduce or minimize modulation of non-targeted tissue (e.g., dorsal column tissue). Various examples disclosed herein are directed to shaping the modulation field to enhance modulation of some neural structures and diminish modulation at other neural structures. The modulation field may be shaped by using multiple independent current control (MICC) or multiple independent voltage control to guide the estimate of current fractionalization among multiple electrodes and estimate a total amplitude that provide a desired strength. For example, the modulation field may be shaped to enhance the modulation of dorsal horn neural tissue and to minimize the modulation of dorsal column tissue. A benefit of MICC is that MICC accounts for various in electrode-tissue coupling efficiency and perception threshold at each individual contact, so that “hotspot” stimulation is eliminated.

The number of electrodes available combined with the ability to generate a variety of complex electrical pulses, presents a huge selection of available modulation parameter sets to the clinician or patient. For example, if the neurostimulation system to be programmed has sixteen electrodes, millions of modulation parameter value combinations may be available for programming into the neurostimulation system. Furthermore, some SCS systems have as many as thirty-two electrodes, which exponentially increases the number of modulation parameter value combinations available for programming.

FIG. 3 illustrates an embodiment of a programming device 302, such as may be implemented in neurostimulation system 100. Programming device 302 represents an embodiment of programming device 102 and includes a storage device 318, a programming control circuit 316, and a user interface device 310. Programming control circuit 316 generates the plurality of stimulation parameters that controls the delivery of the neurostimulation pulses according to the pattern of the neurostimulation pulses. The user interface device 310 represents an embodiment to implement the user interface 110.

In various embodiments, the user interface device 310 includes an input/output device 320 that is capable of receiving user interaction and commands to load, modify, and implement neurostimulation programs and schedule delivery of the neurostimulation programs. In various embodiments, the input/output device 320 allows the user to create, establish, access, and implement respective parameter values of a neurostimulation program through graphical selection (e.g., in a graphical user interface output with the input/output device 320), or other graphical input/output relating to therapy objectives, efficacy of applied treatment, user feedback, and the like. In various examples, the user interface device 310 can receive user input to initiate or control the implementation of the programs or program changes which are recommended, modified, selected, or loaded through use of an open or closed loop programming system, including those driven by freeform text analysis as discussed herein.

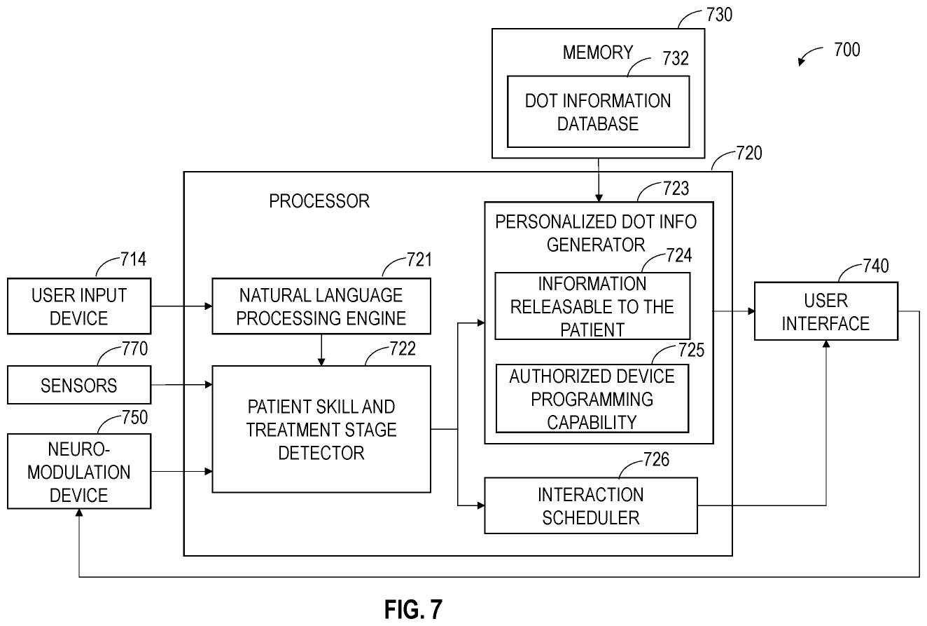

In various embodiments, the input/output device 320 allows the patient user to apply, change, modify, or discontinue certain building blocks of a program and a frequency at which a selected program is delivered. In some embodiments, the input/output device 320 can allow the patient user to save, retrieve, and modify programs (and program settings) loaded from a clinical encounter, managed from the patient feedback computing device, or stored in storage device 318 as templates. In some embodiments, the input/output device 320 can include a display to present to a user (e.g., the patient) customized device operation and treatment information including therapy programming capabilities authorized to the patient. Such information and programming capabilities may be customized in accordance with patient skills or engagement levels with the neuromodulation device and the phases of treatment journey (e.g., before receiving a permanent neuromodulation device, or certain temporal phase or patient state after receiving the device). In some examples, an in-app timeline can be presented to the patient automatically or upon a patient request to keep the patient informed about various treatment options and expected outcomes at current and future treatment phases. By adapting the information presentation and device programming capability to patient skills or engagement levels and treatment phases, enhanced user experience, active patient engagement with their devices and therapies, and more informed exploration of various therapy programs can be realized, which may help patients achieve their treatment goals and improve the overall neuromodulation treatment outcome. Examples of systems and devices that enable customizable interactive presentation of information related to neuromodulation therapy, operation of neuromodulation device, and therapy programming capabilities are discussed below with reference to FIGS. 7, 8A-8B, and 9A-9B.

In various embodiments, the input/output device 320 and accompanying software on the user interface device 310 allows newly created building blocks, program components, programs, and program modifications to be saved, stored, or otherwise persisted in storage device 318. Thus, it will be understood that the user interface device 310 may allow many forms of device operation and control, even if closed loop programming is occurring. The analysis of freeform text, discussed herein, may be in addition to (or in place of) this user input and other forms of closed-loop or open-loop programming.

In one embodiment, the input/output device 320 includes a touchscreen. In various embodiments, the input/output device 320 includes any type of presentation device, such as interactive or non-interactive screens, and any type of user input device that allows the user to interact with a user interface to implement, remove, or schedule the programs. Thus, the input/output device 320 may include one or more of a touchscreen, keyboard, keypad, touchpad, trackball, joystick, and mouse. The logic of the user interface 110, the stimulation control circuit 214, and the programming control circuit 316, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

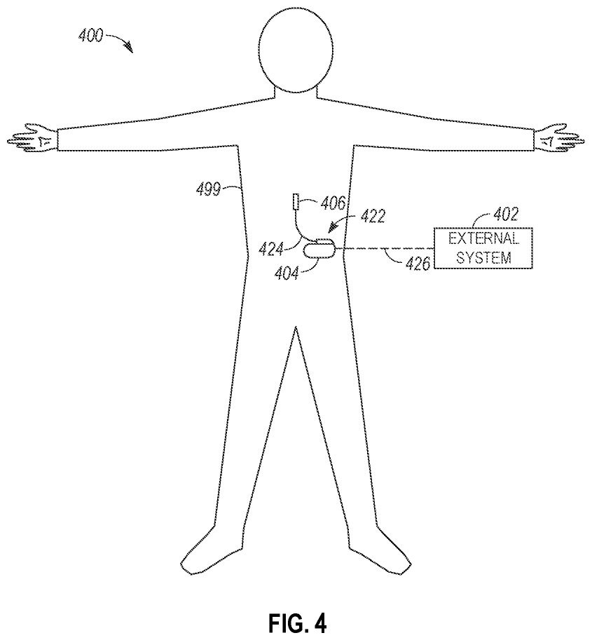

FIG. 4 illustrates an implantable neurostimulation system 400 and portions of an environment in which system 400 may be used. System 400 includes an implantable system 422, an external system 402, and a telemetry link 426 providing for wireless communication between an implantable system 422 and an external system 402. Implantable system 422 is illustrated in FIG. 4 as being implanted in the patient's body 499. The system is illustrated for implantation near the spinal cord. However, the neurostimulation system may be configured to modulate other neural targets.

Implantable system 422 includes an implantable stimulator 404 (also referred to as an implantable pulse generator, or IPG), a lead system 424, and electrodes 406, which represent an embodiment of the stimulation device 204, the lead system 208, and the electrodes 206, respectively. The external system 402 represents an embodiment of the programming device 302.

In various embodiments, the external system 402 includes one or more external (non-implantable) devices each allowing the user and/or the patient to communicate with the implantable system 422. In some embodiments, the external system 402 includes a programming device intended for the user to initialize and adjust settings for the implantable stimulator 404 and a remote control device intended for use by the patient. For example, the remote control device may allow the patient to turn the implantable stimulator 404 on and off and/or adjust certain patient-programmable parameters of the plurality of stimulation parameters. The remote control device may also provide a mechanism to receive and process feedback on the operation of the implantable neurostimulation system. Feedback may include metrics or an efficacy indication reflecting perceived pain, effectiveness of therapies, or other aspects of patient comfort or condition. Such feedback may be automatically detected from a patient's physiological state, collected from other sensors or devices (not shown), or manually obtained from user input entered in a user interface (such as with the user input scenarios discussed below). Such feedback and other information may comprise the device data evaluated as part of association and matching with freeform text input.

As used herein, the terms “neurostimulator,” “stimulator,” “neurostimulation,” and “stimulation” generally refer to the delivery of electrical energy that affects the neuronal activity of neural tissue, which may be excitatory or inhibitory; for example by initiating an action potential, inhibiting or blocking the propagation of action potentials, affecting changes in neurotransmitter/neuromodulator release or uptake, and inducing changes in neuroplasticity or neurogenesis of tissue. It will be understood that other clinical effects and physiological mechanisms may also be provided through use of such stimulation techniques.

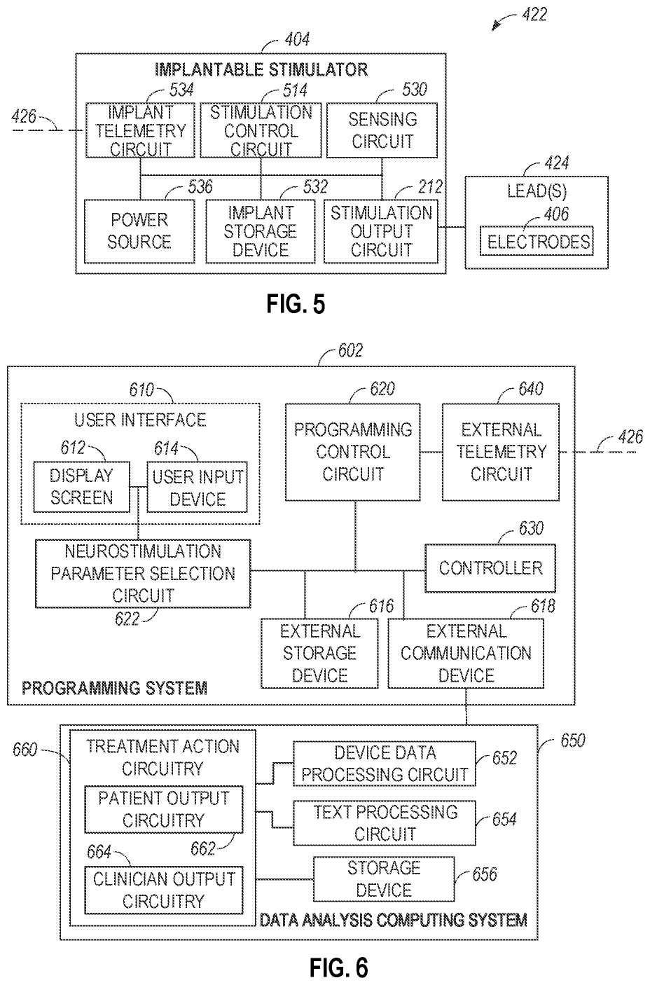

FIG. 5 illustrates an embodiment of the implantable stimulator 404 and the one or more leads 424 of an implantable neurostimulation system, such as the implantable system 422. The implantable stimulator 404 may include a sensing circuit 530 used for an optional sensing capability, stimulation output circuit 212, a stimulation control circuit 514, an implant storage device 532, an implant telemetry circuit 534, and a power source 536. The sensing circuit 530, when included and needed, senses one or more physiological signals for purposes of patient monitoring and/or feedback control of the neurostimulation, including in the closed loop programming processes discussed herein. Examples of the one or more physiological signals includes neural and other signals each indicative of a condition of the patient that is treated by the neurostimulation and/or a response of the patient to the delivery of the neurostimulation.

The stimulation output circuit 212 is electrically connected to electrodes 406 through the one or more leads 424, and delivers each of the neurostimulation pulses through a set of electrodes selected from the electrodes 406. The stimulation output circuit 212 can implement, for example, the generating and delivery of a customized neurostimulation waveform (e.g., implemented from a parameter of a program selected with the closed-loop programming system) to an anatomical target of a patient.

The stimulation control circuit 514 represents an embodiment of the stimulation control circuit 214 and controls the delivery of the neurostimulation pulses using the plurality of stimulation parameters specifying the pattern of the neurostimulation pulses. In one embodiment, the stimulation control circuit 514 controls the delivery of the neurostimulation pulses using the one or more sensed physiological signals and processed input from patient feedback interfaces. The implant telemetry circuit 534 provides the implantable stimulator 404 with wireless communication with another device such as a device of the external system 402, including receiving values of the plurality of stimulation parameters from the external system 402. The implant storage device 532 stores values of the plurality of stimulation parameters, including parameters from one or more programs which are activated, de-activated, or modified using the approaches discussed herein.

The power source 536 provides the implantable stimulator 404 with energy for its operation. In one embodiment, the power source 536 includes a battery. In one embodiment, the power source 536 includes a rechargeable battery and a battery charging circuit for charging the rechargeable battery. The implant telemetry circuit 534 may also function as a power receiver that receives power transmitted from external system 402 through an inductive couple.

In various embodiments, the sensing circuit 530, the stimulation output circuit 212, the stimulation control circuit 514, the implant telemetry circuit 534, the implant storage device 532, and the power source 536 are encapsulated in a hermetically sealed implantable housing. In various embodiments, the lead(s) 424 are implanted such that the electrodes 406 are placed on and/or around one or more targets to which the neurostimulation pulses are to be delivered, while the implantable stimulator 404 is subcutaneously implanted and connected to the lead(s) 424 at the time of implantation.

FIG. 6 illustrates an embodiment of a programming system 602 used as part of an implantable neurostimulation system, such as the external system 402, with the programming system 602 configured to send and receive device data (e.g., commands, parameters, program selections, information). FIG. 6 also illustrates an embodiment of a data analysis computing system 650, communicatively coupled to the programming system 602, with the data analysis computing system 650 used to perform data analysis on freeform text and device data in connection with neuromodulation treatment by the implantable neurostimulation system.

The programming system 602 represents an embodiment of the programming device 302, and includes an external telemetry circuit 640, an external storage device 616, a programming control circuit 620, a user interface device 610, a controller 630, and an external communication device 618, to effect programming of a connected neuromodulation device. The operation of the neurostimulation parameter selection circuit 622 enables selection, modification, and implementation of a particular set of parameters or settings for neurostimulation programming. The particular set of parameters or settings that are selected, modified, or implemented may be based on freeform text analysis.

The external telemetry circuit 640 provides the closed loop programming system 602 with wireless communication to and from another controllable device such as the implantable stimulator 404 via the telemetry link 426, including transmitting one or a plurality of stimulation parameters (including selected, identified, or modified stimulation parameters of a selected program) to the implantable stimulator 404. In one embodiment, the external telemetry circuit 640 also transmits power to the implantable stimulator 404 through inductive coupling.

The external communication device 618 may provide a mechanism to conduct communications with a programming information source, such as a data service, program modeling system, to receive program information, settings and values, models, functionality controls, or the like, via an external communication link (not shown). In a specific example, the external communication device 618 communicates with the data analysis computing system 650 to obtain commands or instructions in connection with parameters or settings that are selected, modified, or implemented based on freeform text analysis from the data analysis computing system 650. The external communication device 618 may communicate using any number of wired or wireless communication mechanisms described in this document, including but not limited to IEEE 802.11 (Wi-Fi), Bluetooth, Infrared, and like standardized and proprietary wireless communications implementations. Although the external telemetry circuit 640 and the external communication device 618 are depicted as separate components within the closed-loop programming system 602, the functionality of both of these components may be integrated into a single communication chipset, circuitry, or device.

The external storage device 616 stores a plurality of existing neurostimulation waveforms, including definable waveforms for use as a portion of the pattern of the neurostimulation pulses, settings and setting values, other portions of a program, and related treatment efficacy indication values. In various embodiments, each waveform of the plurality of individually definable waveforms includes one or more pulses of the neurostimulation pulses, and may include one or more other waveforms of the plurality of individually definable waveforms. Examples of such waveforms include pulses, pulse blocks, pulse trains, and train groupings, and programs. The existing waveforms stored in the external storage device 616 can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

The external storage device 616 may also store a plurality of individually definable fields that may be implemented as part of a program. Each waveform of the plurality of individually definable waveforms is associated with one or more fields of the plurality of individually definable fields. Each field of the plurality of individually definable fields is defined by one or more electrodes of the plurality of electrodes through which a pulse of the neurostimulation pulses is delivered and a current distribution of the pulse over the one or more electrodes. A variety of settings in a program may be correlated to the control of these waveforms and definable fields.

The programming control circuit 620 represents an embodiment of a programming control circuit 316 and may translate or generate the specific stimulation parameters or changes which are to be transmitted to the implantable stimulator 404, based on the results of the neurostimulation parameter selection circuit 622. The pattern may be defined using one or more waveforms selected from the plurality of individually definable waveforms (e.g., defined by a program) stored in an external storage device 616. In various embodiments, the programming control circuit 620 checks values of the plurality of stimulation parameters against safety rules to limit these values within constraints of the safety rules. In one embodiment, the safety rules are heuristic rules.

The user interface device 610 represents an embodiment of the user interface device 310 and allows the user (including a patient or clinician) to provide input relevant to therapy objectives, such as to switch programs or change operational use of the programs. The user interface device 610 includes a display screen 612, a user input device 614, and may implement or couple to the parameter selection circuit 622, or data provided from the data analysis computing system 650. The display screen 612 may include any type of interactive or non-interactive screens, and the user input device 614 may include any type of user input devices that supports the various functions discussed in this document, such as a touchscreen, keyboard, keypad, touchpad, trackball, joystick, and mouse. The user interface device 610 may also allow the user to perform other functions where user interface input is suitable (e.g., to select, modify, enable, disable, activate, schedule, or otherwise define a program, sets of programs, provide feedback or input values, or perform other monitoring and programming tasks). Although not shown, the user interface device 610 may also generate a visualization of such characteristics of device implementation or programming, and receive and implement commands to implement or revert the program and the neurostimulator operational values (including a status of implementation for such operational values). These commands and visualization may be performed in a review and guidance mode, status mode, or in a real-time programming mode. Similar to the interface device 310 as described above, the user interface device 610 can display on the display screen 612 customized device and treatment information and customized therapy programming capabilities authorized to the patient. Such presentation of information and therapy programming capabilities are customized in accordance with patient skills or engagement levels with their neuromodulation devices and/or the phases of treatment journey. In some examples, an in-app timeline can be displayed automatically or upon patient request to keep the patient informed about various treatment options and expected outcomes at current and future treatment phases. The patient may use the input device 614 to provide input including, for example, inquiries about device functions or operations, treatment operations available for them to use, expectations of treatment outcome, personal treatment objectives or intended usage of the neuromodulation device, feedback on treatment, etc. The input or feedback from the patient can be provided in response to or interactively with the presentation of the customized device and treatment information and customized therapy programming capabilities. The user input or feedback may be processed by the data analysis computing system 650.

The controller 630 can be a microprocessor that communicates with the external telemetry circuit 640, the external communication device 618, the external storage device 616, the programming control circuit 620, the parameter selection circuit, and the user interface device 610, via a bidirectional data bus. The controller 630 can be implemented by other types of logic circuitry (e.g., discrete components or programmable logic arrays) using a state machine type of design. As used in this disclosure, the term “circuitry” should be taken to refer to discrete logic circuitry, firmware, or to the programming of a microprocessor.

The data analysis computing system 650 is configured to operate treatment action circuitry 660, which may produce or initiate certain actions on the basis of device data (received and processed by device data processing circuit 652) and freeform input text or voices (received and processed by text processing circuit 654). The treatment action circuitry 660 may identify one or more actions related to the neuromodulation treatment, and provide outputs to a patient or a clinician using patient output circuitry 662 or clinician output circuitry 664, respectively. Such outputs and actions provided by the outputs are based on the evaluation and detection of particular patient states and device states from freeform text and associated device data, discussed in more detail below.

The data analysis computing system 650 also is depicted as including a storage device 656 to store or persist data related to the device data, freeform text input, patient or clinician output, and related settings, logic, or algorithms. Other hardware features of the data analysis computing system 650 are not depicted for simplicity, but are suggested from functional capabilities and operations in the following figures.

As will be understood, patients who are experiencing chronic pain are often willing to provide detailed information regarding their current medical state, treatment or physical objectives using freeform texts either voluntarily or prompted with questions. Freeform text in the form of a narrative, explanatory statement, or interjection is easy for patients to produce, and can provide many details regarding a patient's actions, physiological and physiological state, prior historical events, treatment and physical objectives, desired operation mode or a habit of usage of the neuromodulation device, and can reflect both objective and subjective results of neuromodulation treatment. Freeform text, however, can be time-consuming or difficult for physicians and clinicians to interpret, especially when patient feedback may be contradictory. The systems and methods described in this document in accordance to various embodiments can more efficiently and quickly interpret patient text, determine a patient state based on the interpreted patient text, produce useful outcomes for diagnosis, treatment, and remediation relevant to neuromodulation device operation.

FIG. 7 illustrates, by way of example, an assisted therapy programming system 700 for providing customizable interactive healthcare services in relation to neuromodulation treatment via a neuromodulation device. The assisted therapy programming system 700, which is an embodiment of at least a portion of the neurostimulation system 400, can include a processor 720 configured to determine customized information about the neuromodulation device and treatment releasable to the patient and/or customized therapy programming capabilities authorized to the patient. The processor 720 can further customize the delivery of said information and programming capabilities to the patient. Such customized healthcare services can be provided in accordance with patient skill or engagement levels with their neuromodulation devices and the phases of treatment journey. The assisted therapy programming system 700 can assist patients in using and tuning neuromodulation therapies to meet their treatment goals in a more convenient and effective manner.

The processor 720 can determine customized device and treatment information to be provided to a patient and/or the device programming capability authorized to the patient based on input from one or more of a user input device 714, patient sensors 770, or a neuromodulation device 750. The user input device 714, which is an embodiment of the user input device 614, can be in a form of a computing device (e.g., a laptop computer, a tablet, or a smartphone, among other user-interactive devices) that includes a graphical user interface and text input functionality that enables information exchange with a patient. The text input functionality may receive freeform text from a patient via questionnaires, surveys, messages, or other textual inputs. Although not depicted, other forms of non-text input functionality may also be provided.

The freeform text may be received using a chatbot functionality or a messaging functionality provided by the user input device 714 (e.g., apps in a smartphone). For example, the user may use the user input device 714 to provide answers to a questionnaire or an interactive questions and answers (e.g., an automated chatbot session) on the user interface. The text content may indicate or relate to pain or satisfaction, physiological or emotional state of the patient, QoL attributes, responses to neuromodulation treatment, among others. In specific examples, the text content originates from at least one of: text provided in a text chat session (e.g., transcript text) conducted between a chatbot and the patient; a voice chat session conducted between a virtual agent and the patient, with at least a portion of the voice chat session converted to text (e.g., a transcript of the chat session conversation); a text message session conducted between a text service and the patient (e.g., a transcript of one or more SMS text conversations); or an audio recording of a discussion conducted between the patient and a human agent, with at least a portion of the audio recording converted to text (e.g., a transcript of the audio recording); or a freeform text input provided by the patient (e.g., survey or question responses, narrations, etc.). In some examples, the user input device 714 may include a programming functionality to provide one or more outputs in the graphical user interface related to programming control or implementation. The programming functionality specifically may provide the patient with therapy content and programming recommendations generated by the data analysis computing system 650. Other form factors and interfaces such as audio interfaces and text interfaces may also be substituted for or augmented with the user input device 714.

The processor 720 can include a natural language processing (NLP) engine 721 to analyze the freeform text input received from the user input device 714. The analysis of text input may occur using one or more forms of text parsing, linguistic analysis, topic modeling, etc. In an example, an algorithm provided by the NLP engine can translate the patient freeform textual interactions to valence (polarity) scores. These polarity scores may represent a negative or positive sentiment (e.g., in a range from values −1 to +1), or an intensity of positive or negative sentiment, captured at or associated with a particular time. The polarity scores can be cross-referenced against device data (e.g., program usage, device on/off state, physiological state from a sensor, etc.), and the polarity of a particular text statement may be directly determined as a result of sentiment analysis performed using any number of NLP techniques. The polarity of a text statement and the resulting patient state may be used, for instance, to identify the most effective settings of a neurostimulation program, directly from patient feedback and responses collected over time. In another example, an NLP algorithm can translate the patient freeform textual interactions to specific device diagnostics to be initiated at specific time, such as to evaluate various aspects of device data from the neuromodulation device 750. This may include, checking current battery level, identifying a current program, identifying device impedance, verifying program settings, performing logging or evaluation of logging information, initiating troubleshooting procedures, and the like. U.S. Provisional Patent Application 63/287,828 provides a detailed disclosure of systems and methods for interpreting patient text or voice input, and producing useful outcomes for diagnosis, treatment, and remediation relevant to neuromodulation therapy and device operation, which are incorporated herein by reference in its entirety.

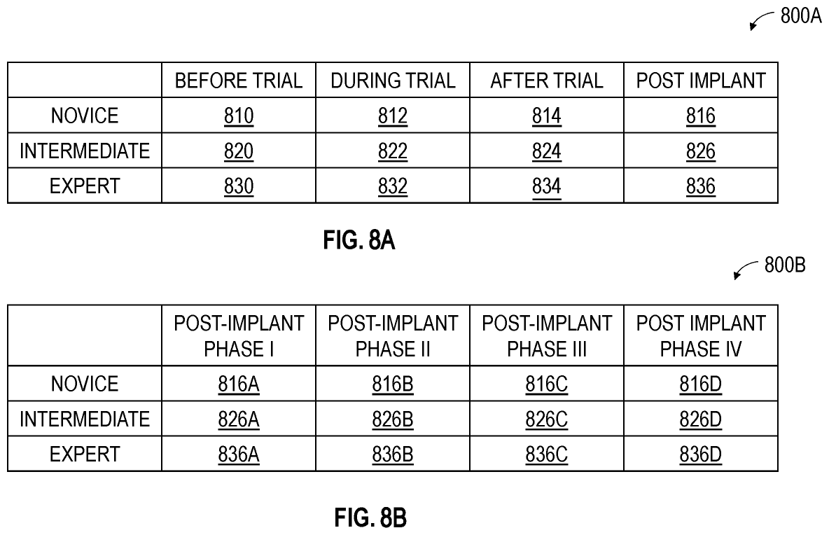

The processor 720 can include a patient skill and treatment stage detector 722 that can determine a treatment phase (or treatment stage) and/or to identify a skill or engagement level of the patient at the treatment phase. A treatment phase can be determined as a time period relative to a date of a medical event or a predefined milestone, such as enrollment in a device trial (such as a minimally invasive SCS trial to determine future success of implantation of a permanent neurostimulator device), termination of the device trial, implantation of a permanent implantable neuromodulation device, one-month mark post implantation, 3-month mark post implantation, one-year mark post implantation, etc. The treatment phase may additionally or alternatively be represented by a stage of patient experience with a neuromodulation therapy after receiving the neuromodulation device. A patient skill or engagement level represents patient knowledge level towards disease or medical conditions being treated (e.g., chronic pain), device functionalities and treatment options offered by the neuromodulation device, and patient skills, experiences, and comfort levels of interacting with the neuromodulation device (e.g., adjusting neurostimulation parameters or exploring different therapy programs). Examples of treatment phases and patient skill or engagement levels are discussed further with reference to FIGS. 8A-8B.

The patient skill and treatment stage detector 722 can determine a treatment phase and/or identify a skill or engagement level of the patient at the identified treatment phase based on the freeform text input from the patient via the user input device 714 and processed by the NLP engine 721. In an example, the NLP engine 721 can interpret the patient freeform text and make inference about patient knowledge, skills, or engagement levels with respect to neuromodulation therapy or neuromodulation device based on the content of the text input or the manner in which the text input is provided. For example, if the patient provides the freeform texts “What does SCS do?”, “How to I charge my device?”, or “How do I turn the device OFF?”, then the NLP engine 721 can use topic modeling to identify words or a group of words that would categorize the patient into a “novice” track of skills or engagement levels. In another example, if the patient provides freeform text in relation to more advanced or complex topics of device operation or therapy programming, such as “I want to change the stimulation to my upper right back”, or “I would like to switch to a different stimulation program during sleep”, then the NLP engine 721 can identify the words that would categorize the patient into an “intermediate” or an “expert” track of skills and engagement levels.

In some examples, the patient skill and treatment stage detector 722 can track patient interactions with the neuromodulation device over time, and identify the patient skill or engagement level based on the tracked interactions. For example, the patient skill and treatment stage detector 722 can track the patient's pattern or frequency of searching for information about device functions or therapies options, or making attempts to adjust an device setting (e.g., adjusting a therapy parameter). The tracked pattern or frequency of device usage defines a device usage persona for the patient. Examples of the patient device usage persona may include: never exploring other therapy programs or make an attempt to change a device parameter; frequent attempts to change stimulation programs; maintaining a consistent pattern of usage; or making a one-time search for an optimal program then making no change for a sustained period of time. The patient skill and treatment stage detector 722 can categorize the patient into one of a “novice”, an “intermediate”, or an “expect” track of skills or engagement levels based on patient device usage persona. In some examples, the personalized DOT information generator 723 may use the patient device usage persona to generate personalized recommendations to the patient to make necessary adjustment of how they interact with the device, tune therapy parameters or explore other therapy programs, or make lifestyle changes.

In some examples, the patient or an authorized user (e.g., the physician) can manually select or update the skill or engagement level for the patient. In some examples, a diagnostic test of patient skill or engagement levels may be performed automatically or on demand, and the patient skill and treatment stage detector 722 can determine a treatment phase and/or identify a skill or engagement level of the patient based on the results of the diagnostic test. During the diagnostic test, the patient may be requested to perform one or more tasks of different complexities to interact with the neuromodulation device, such as adjusting a device parameter values. Patient skill or engagement level can be assessed based on, for example, patient responsiveness to the command and performance of completing the requested tasks. If the patient responds to the command promptly (within a preset time period) and successfully completes a task of medium-level complexity such as increasing the pulse amplitude, then the patient can be categorized into the “intermediate” track. If the patient responds promptly and successfully completes a more complex task such as switching to a different stimulation program or programming for multi-area stimulation, then the patient can be categorized into the “expert” track. If the patient does not respond to the command within the preset time or fail to complete the requested task, then the patient can be categorized into the “novice” track.

In addition or alternative to the processed freeform text input from the patient, the patient skill and treatment stage detector 722 can determine the treatment phase and/or to identify a skill or engagement level of the patient using data collected by one or more patients sensors 770 and/or data collected by the neuromodulation device 750 (when available, such as during any post-implant phases). The neuromodulation device 750 is an embodiment of the stimulation device 104 or 204, or the implantable stimulator 404. In an example, the neuromodulation device 750 is an implantable neurostimulator system including a pulse generator and associated lead and electrodes configured to provide spinal cord stimulation (SCS) for treating or alleviating chronic pain or other medical conditions. The neuromodulation device 750 may include one or more sensors to sense physiological or functional signals or patient responses to therapies delivered to the patient. The patient sensors 770 may include wearables, sleep trackers, motion tracker, implantable monitors, etc. The patients sensors 770 and/or the neuromodulation device 750 can each collect physiological or functional data from the patient, which may indicate occurrence of medical events or milestones that are used for defining the treatment phase.

In some examples, depending on the treatment phase, a patient may be defaulted into a particular skill or engagement level. For example, a patient presumably has little knowledge about the device and neuromodulation treatment during the pre-implant trial period, or further within the one-month post-implant period, and can thus be defaulted into the “novice” track during that time. As the patient advances to later phases along the treatment journey, the patient is presumably more familiar with the device functions and treatment options, and thus can be defaulted into a higher skill or engagement level such as an “intermediate” track or an “expert” track. The patient skill and treatment stage detector 722 can modify the default skill or engagement level automatically or on demand (e.g., per request by the patient or clinician).

The processor 720 can include a personalized device operation and treatment (DOT) information generator 723 configured to generate personalized DOT information for the patient based on the treatment phase and the patient skill or engagement level with the neuromodulation device. The personalized DOT information can include information releasable to the patient 724, which may include automatic or on-demand presentation of information about the diseases or the medical condition to be treated, device information and operation instructions, device functions, neuromodulation therapy options, trackable treatment goals, etc. The personalized DOT information may additionally or alternatively include authorized device programming capability 725, which may include authorized selection or adjustment of stimulation parameters or programs. By adapting the information presentation and patient device programming capability to treatment phase and patient skill or engagement levels, device and treatment information and the device programming capability may vary from patient to patient depending on their skills or engagement levels. The same patient may receive different device and therapy information and have different capabilities of programming a stimulation parameter during different stages of treatment journey.

The system 700 includes a memory 730 that stores and maintains a database 732 of DOT information with respect to the neuromodulation device. The database of DOT information can be categorized according to a plurality of pre-determined treatment phases and a plurality of pre-determined patient skill or engagement levels with the neuromodulation device. To determine personalized DOT information for a patient at a particular treatment phase, the personalized DOT information generator 723 can query the database 732 to identify personalized DOT information for the patient that corresponds to the treatment phase and the skill or engagement level of the patient as identified by the patient skill and treatment stage detector 722.

Referring to FIGS. 8A-8B which illustrate non-limiting examples of the DOT information database 732, the DOT information is categorized in a two-dimensional data array, depicted in a table format. In particular, FIG. 8A illustrates a table 800A of DOT information categorized according to a plurality of pre-determined patient skills or engagement levels and a plurality of pre-determined phases in a treatment journey. The pre-determined patient skills or engagement levels include a “novice” track, an “intermediate” track, and an “expert” track, representing different levels of cognitive performances, knowledge, experiences, and skills with the neuromodulation device, general inclination of using or degrees of engagement with therapies offered by the device. The pre-determined patient skills or engagement levels can additionally or alternatively be represented by a plurality of pre-determined patterns of patient interaction with the neuromodulation device to adjust a neuromodulation therapy parameter, also referred to as device usage personae. Examples of the device usage personae may include: never exploring other therapy programs or make an attempt to change a device parameter; frequent attempts to change stimulation programs; maintaining a consistent pattern of usage; or making a one-time search for an optimal program then making no change for a sustained period of time.

The pre-determined treatment phases can include temporal phases representing respective times elapsed from a specific medical event or a predefined milestone. By way of example, table 800A includes a “before trial” phase representing a time period before patient enrollment in a device trial involving treatment provided by a temporary or experimental device; a “during trial” phase representing a time period between the enrollment and termination of the trial for the purpose of determining patient candidacy for implantation of a permanent device; a “after trial” phase representing a time period after the trial but prior to receiving a permanent medical device such as an implantable neuromodulation device; and a “post-implant” phase representing a time period after receiving a permanent device.

Different DOT information may be provided to a patient based on the treatment phase and the patient's skill or engagement level. For example, a patient in the “novice” track can access DOT information 810 before trial, 812 during trial, 814 after trial, and 816 post implant; a patient in the “intermediate” track can access DOT information 820 before trial, 822 during trial, 824 after trial, and 826 post implant; a patient in the “expert” track can access DOT information 830 before trial, 832 during trial, 834 after trial, and 836 post implant. The DOT information in each category (as defined by one of the skill or engagement levels at one of the treatment phases) includes information releasable to the patient (e.g., diseases or the medical condition to be treated, device information and operation instructions, device functions, neuromodulation therapy options, trackable treatment goals, etc.) and device programming capability authorized to the patient at that treatment phase (e.g., authorized selection or adjustment of stimulation parameters or programs). The DOT information in one category can differ from the DOT information in a different category in the amount or the type of information releasable to the patient, and/or the device programming capability authorized to the patient. For example, the DOT information 826 for a patient in a “intermediate” track at “post implant” phase may include a higher volume or more advanced information on device operation and treatment, or more complex therapy programming capabilities authorized to the patient than the DOT information 812 for a patient of “novice” track at “during trial” phase. By way of example, the DOT information provided to the patient at different treatment phases can include: (i) for the “before trial” phase, information about the SCS, resources or contacts available to the patient regarding the device trial; (ii) for the “during trial” phase, information about the SCS therapies used in the trial, what to expect during the treatment, and assessment whether the treatment works (e.g., pain relief); (iii) for the “after trial” phase, information about permanent neuromodulation device or other treatment options, candidacy for an implantable neuromodulation device, preparation for receiving an implantable neuromodulation device; (iv) for the “post implant” phase, educational information for the device received, device operation guide, therapy options; differences and functions provided by different neurostimulation programs, how to program or select a program, when to switch to a different program, when to ask for new programs; device usage instructions such as how to effectively charge the neuromodulation device, how to handle automatic stimulation change, etc.

As discussed above, the categorized DOT information at different treatment phases may vary in accordance with patient skill or engagement levels with the device. For example, the device programming capability authorized to the patient at a particular treatment stage may vary according to the patient skills or engagement levels. In some examples, patients in the “novice” track are authorized to make relatively basic device operations such as turning on or off the device (to continue or temporarily withhold neuromodulation therapy, use up/down arrows to select parameter values); patients in the “intermediate” track are authorized to make more complicated operations such as changing a series of stimulation parameters such as pulse amplitude, pulse width, stimulation frequency, reconfigure stimulation delivery to provide area-specific treatment (e.g., switch to a different stimulation area); patients in the “expert” track are authorized to make advanced device operations and treatment optimization such as switching between different stimulation programs.

In some examples, a treatment phase (such as that shown in FIG. 8A) can be further broken down into a number of sub-phases, and respective DOT information may be assigned thereto. FIG. 8B illustrates a table 800B of DOT information categorized for a plurality of pre-determined skill or engagement levels (e.g., a “novice” track, an “intermediate” track, and an “expert” track as described above with respect to table 800A) and for a plurality of pre-determined post-implant phases I, II, III, and IV. In an example, the post-implant phases include temporal phases representing respective times elapsed from the date of receiving the neuromodulation device (e.g., implantation), such as one to two weeks for post-implant phase I, two weeks to three months for post-implant phase II, three months to one year for post-implant phase III, and beyond one year for post-implant phase IV. According to table 800B, a patient in the “novice” track can access DOT information 816A, 816B, 816C, and 816D at post-implant phases I, II, III, and IV, respectively; a patient in the “intermediate” track can access DOT information 826A, 826B, 826C, and 826D at post-implant phases I, II, III, and IV, respectively; a patient in the “expert” track can access DOT information 836A, 836B, 836C, and 836D at post-implant phases I, II, III, and IV, respectively. Similar to the description above with respect to table 800A, the DOT information in each category (as defined by one of the skill or engagement levels at one of the post-implant treatment phases) includes information releasable to the patient and device programming capability authorized to the patient. The DOT information in one category can differ from the DOT information in a different category in the amount or the type of information releasable to the patient, and/or the device programming capability authorized to the patient. By way of example, the DOT information provided to the patient at different post-implant treatment phases can include: (i) for post-implant phase I, information about basic implantable system usage, basic charging instructions, effect of posture or physical activities on neurostimulation therapy, a higher volume or more frequent patient interactions (e.g., patient status check) via a user interface (e.g., weekly patient interactions); (ii) for post-implant phase II, information about and detection of migration of lead delivering neuromodulation therapy, setting personalized goal; (iii) for post-implant phase III, tracking progress toward the personalized treatment goal, a reduced volume or less frequent patient interactions (e.g., monthly patient interaction); (iv) for post-implant phase IV, information on new products, new therapy programs available, new services provided, new educational materials available, query patient health status change or progression of the disease.

In addition or alternative to the temporal phases as described above, the post-implant phases may be represented by different stages of patient needs or experiences with neuromodulation therapies. Unlike the temporal phases, the patient need or experience-based post-implant stages generally do not have preset durations, and may not follow the same sequential order. A patient may transition back and forth between different post-implant stages as the underlying disease or condition changes, or as new therapies become available, etc. By way of example, post-implant phase I can be a “trial and error” stage during which new therapy programs are added or patient experiences changes in condition (e.g., changes in underlying pain), and the patient would experiment different therapy programs until one is found to provide desired outcome; post-implant phase II can be a “steady state” stage during which an optimal or a desired therapy program is found, and the patient would keep using said therapy program without substantial interaction with the neuromodulation device (e.g., no or few parameter adjustments); and post-implant phase III can be a “help needed” stage where the therapy program does not appear working or patient condition worsens, yet existing therapy options have been exhausted). The DOT information provided to the patient at different post-implant treatment phases can be different. For example, simpler information can be provided for “steady state,” while more complex information can be provided for “help needed” phase. A higher volume or more frequent user interactions can be provided for the “help needed” phase than for the “steady state” phase.

Referring back to FIG. 7, the personalized DOT information generator 723 can query the database 732 to search for a stored pre-determined DOT information with the corresponding treatment phase and the skill or engagement level that match respectively the treatment phase and the skill or engagement level for the patient as determined by the patient skill and treatment stage detector 722. Such pre-determined DOT information (graphically represented as a table entry in table 800A or table 800B) is also referred to “matched DOT information.” Device and treatment information releasable to patient 724 and the authorized device programming capability 725 can then be determined from the matched DOT information. The information releasable to patient 724 and the authorized device programming capability 725 can be presented to the patient on a user interface 740, such as a user interface on a smartphone. In some examples, the user interface 740 may be communicatively coupled to the neuromodulation device 750, and the patient may adjust stimulation parameter values, select stimulation programs, or change device setting of the neuromodulation device 750 in accordance with the authorized device programming capability 725 via the user interface 740.

The processor 720 may include an interaction scheduler 726 that can determine a personalized mode of interaction with the patient in accordance with the determined treatment phase and the skill or engagement level of the patient. While the personalized DOT information generator 723 determines what type and/or amount of information is releasable to the patient and what device programming capabilities are to authorized to the patient, the interaction scheduler 726 can determine how the personalized DOT information is to be provided to the patient. The personalized mode of interaction can include, for example, timing, a rate (frequency), a pattern, or a schedule of presenting the personalized DOT information to the patient. For example, presentation or interactive question and answer sessions (e.g., an automated chatbot session) can be initiated and provided to a patient with a lower level of device knowledge and operating skills at a higher frequency (e.g., once a day) than to a patient having a higher level of knowledge and operating skills (e.g., once a week). In another example, more frequent presentation or interactive question and answer sessions may be given to patients with a higher level of engagement with the device and neuromodulation therapies than those with lower engagement levels.

In various examples, the patient skill and treatment stage detector 722 can detect a change in treatment phase, or a change in patient skill or engagement level. The detection of such change can be performed periodically or in response to a user (e.g., the patient) request. The patient skill and treatment stage detector 722 can update the query of the database 732 to determine updated personalized DOT information for the patient in accordance with the new treatment phase or the patient skill or engagement level, and interactively present the updated personalized DOT information on the user interface 740. This will ensure timely delivery of customized device and therapy information and authorized device programming capability to the patient.

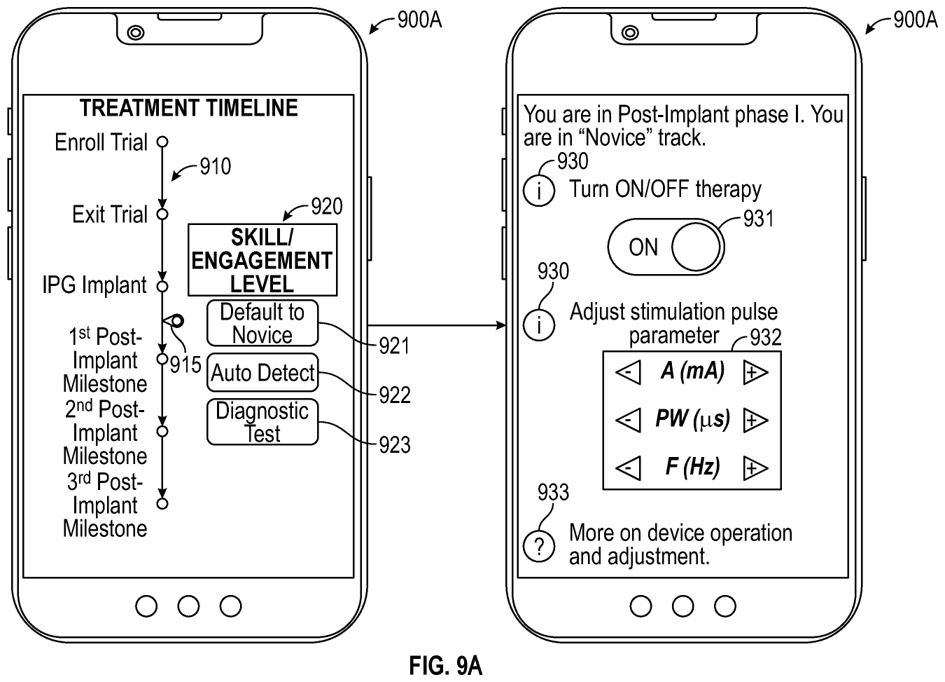

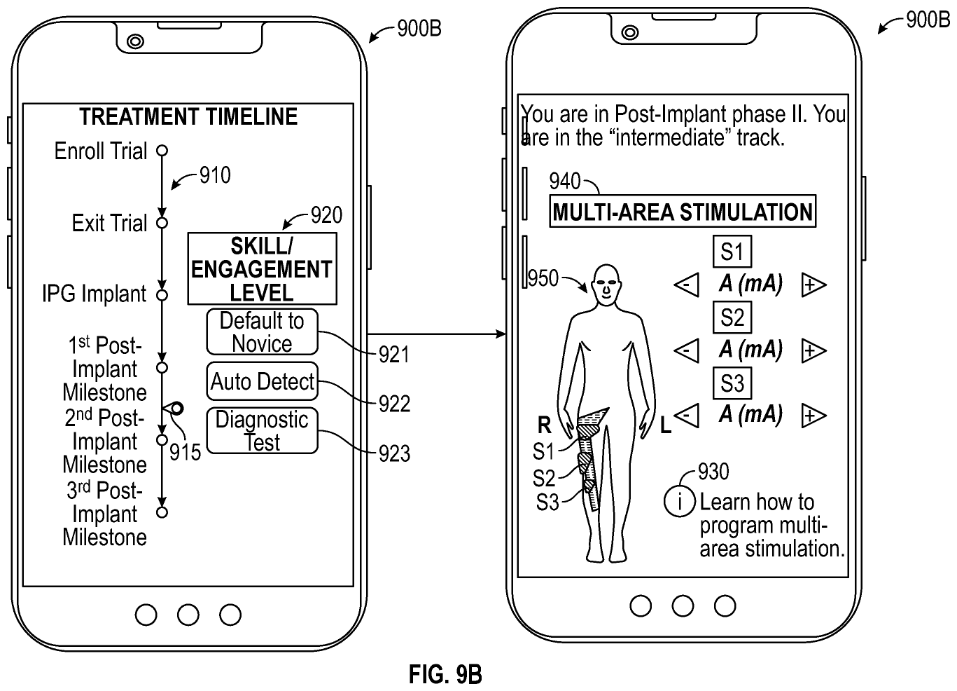

FIGS. 9A-9B illustrate, by way of example and not limitation, user interfaces on a patient programmer (e.g., a smartphone) that provides customized interactive presentation of information about the neuromodulation device and treatment to the patient in accordance with the skill or engagement level of the patient and the treatment phase. The user interface also allows the patient to adjust certain stimulation parameters or device settings using authorized device programming capability that can be determined based on the patient's skill or engagement level and the treatment phase, as described above with reference to FIG. 7. In addition to the customizable contents of the information being presented, the user interface can have customizable features (e.g., UI control elements) that facilitate presentation of the customized DOT information.

FIG. 9A illustrates a user interface 900A depicting an in-app timeline 910 of treatment phases, along with an indication of the patient's present treatment phase, which is represented by a location icon 915 in this example. The patient present treatment phase may be detected by the patient skill and treatment stage detector 722. Alternatively, the patient may identify a proper treatment phase on the timeline 910 such as by sliding and positioning the location icon 915 at a proper location on the timeline 910. The user interface 900A further depicts a patient skill or engagement level menu 920, which allows the patient to determine or confirm his or her skill or engagement level using one of the available options. The “default to novice” option 921, when activated (e.g., tapped), can automatically categorize the patient into “novice” track. The “auto detect” option 922, when activated, can trigger the patient skill and treatment stage detector 722 to automatically detect patient skill or engagement level. The “diagnostic test” option 923, when activated, can trigger a diagnostic test of patient skill or engagement levels, as described above with reference to FIG. 7.

In accordance with the automatically detected or user identified treatment phase and the skill or engagement level, the personalized DOT information generator 723 can query the DOT information database 732 to determine customized device and treatment information and programming capabilities authorized to the patient, and the user interface 900A may present the customized information to the patient. In the illustrated example, the patient is at post-implant phase I, and defaulted to “novice” track. Accordingly, only basic device and therapy information is released to the patient, and fundamental device programming capabilities are authorized to the patient. By way of example, the programming capabilities authorized to a patient in a “novice” level at the post-implant phase I include turning on or off the device 931, and moving arrows to adjust simple parameter values 932. The patient may access educational materials regarding the device operation information such as instructions on how to perform the authorized programming operations via the information icon 930 (e.g., by hovering the mouse over or clicking on the icon 930). The user interface 900A may further allow the patient to explore additional device and therapy information or ask questions using the question button 933.

As the patient advances to a higher skill or engagement level and/or enters into a different post-implant phase, the device and treatment information and programming capabilities authorized to the patient can be changed automatically. FIG. 9B illustrates a user interface 900B showing that the patient is categorized into the “intermediate” track, and the treatment progresses into post-implant phase II. Accordingly, the personalized DOT information generator 723 can query the DOT information database 732 database using the new treatment phase and skill or engagement level information, and determine customized device and treatment information and programming capabilities authorized to the patient. Additional device or therapy information can be released to the patient, and more complex device programming capabilities are authorized to the patient. By way of example, corresponding to the “intermediate” level at the post-implant phase II, the programming capabilities include programming multi-area stimulation 940 that allows the patient to change amplitude per area for a multiarea stimulation program. A pain map 950 can be shown on the user interface 900B, which allows the patient to identify (e.g., using a UI control element) pain areas to be stimulated with different parameter values, such as user-identified pain areas S1, S2, S3 on the pain map 950. The patient may access educational materials regarding the device operation information such as instructions on how to perform multiarea stimulation by hovering the mouse over or clicking on the icon 930.

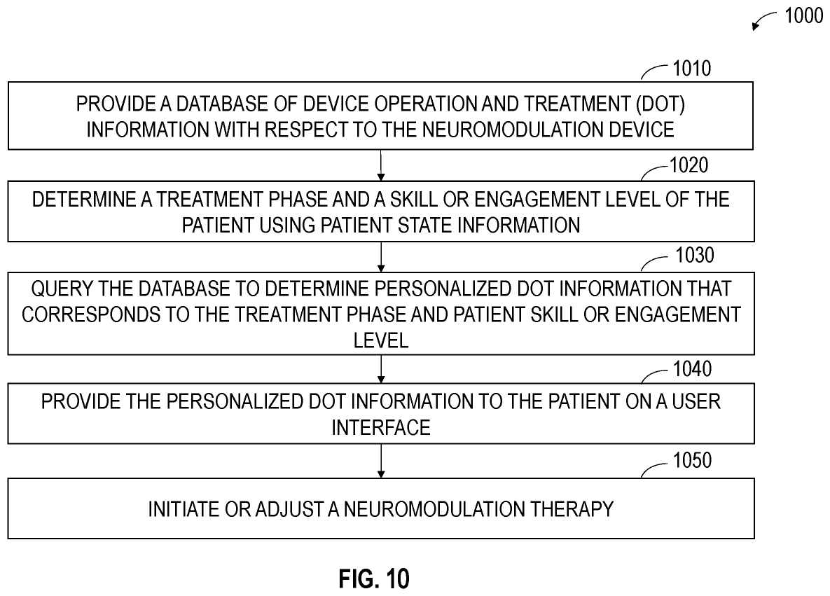

FIG. 10 illustrates, by way of example and not limitation, a method 1000 for providing customizable interactive healthcare services to a patient in relation to neuromodulation treatment via a neuromodulation device, such as the neuromodulation device 750. The method 1000 can be implemented in and carried out by the assisted therapy programming system 700.

At 1010, a database of device operation and treatment (DOT) information with respect to a neuromodulation device can be provided or created. The database of DOT information can be categorized according to a plurality of pre-determined treatment phases and a plurality of pre-determined patient skills or engagement levels with the neuromodulation device. As described above with reference to FIGS. 8A-8B, the DOT information in each category (as defined by one of the skill or engagement levels at one of the treatment phases) can include information releasable to the patient, such as diseases or the medical condition to be treated, device information and operation instructions, device functions, neuromodulation therapy options, trackable treatment goals, etc. The DOT information in each category can include device programming capability authorized to the patient at that treatment phase, such as authorized selection or adjustment of stimulation parameters or programs. The DOT information in one category can differ from the DOT information in a different category in the amount or the type of device and therapy information releasable to the patient, and/or the device programming capability authorized to the patient. For example, between a first skill or engagement level and a second skill or engagement level lower than the first skill or engagement level, the DOT information corresponding to the first (higher) skill or engagement level can include a higher volume or more advanced device and therapy information releasable to the patient than the DOT information corresponding to the second skill or engagement level. Additionally or alternatively, the authorized device programming capability corresponding to the first skill or engagement level includes more advanced device programming capabilities than the device operation and treatment programming capabilities corresponding to the second skill or engagement level.

The pre-determined patient skills or engagement levels can be represented by different levels of cognitive performances, knowledge, experiences, and skills with the neuromodulation device, general inclination of using or degrees of engagement with therapies offered by the device, etc. By way of example and as illustrated in FIGS. 8A-8B, the pre-determined patient skills or engagement levels can include a “novice” track, an “intermediate” track, and an “expert” track. Additionally or alternatively, the pre-determined patient skills or engagement levels can be represented by a plurality of pre-determined patterns of patient interaction with the neuromodulation device to adjust a neuromodulation therapy parameter, also referred to as device usage personae. The pre-determined treatment phases can include temporal phases representing respective times elapsed from a specific medical event or a predefined milestone, such as enrollment in a clinical trial, termination of the clinical trial, implantation of a permanent implantable neuromodulation device, one-month mark post implantation, 3-month mark post implantation, one-year mark post implantation, etc. Additionally or alternatively, the pre-determined treatment phases can be represented by a stage of patient experience with a neuromodulation therapy after receiving the neuromodulation device, as described above with reference to FIGS. 8A-8B.

At 1020, a treatment phase and a skill or engagement level can be determined for a patient scheduled to receive or having received a neuromodulation device, such as using the patient skill and treatment stage detector 722. Various sources of information may be used to determine the treatment phase and the patient's skill or engagement level. In an example, the patient's text or voice input can be received by the user input device 714, processed using natural language processing (NLP) methods via the NLP engine 721, and used to determine the treatment phase and the patient's skill or engagement level. In some examples, patient interactions with the neuromodulation device can be tracked over time, and the patient skill or engagement level can be identified based on the tracked interactions. In another example, a diagnostic test of patient skill or engagement levels may be performed automatically or on demand, and the treatment phase and the patient skill or engagement level can be determined based on the results of the diagnostic test. In an example, patient or an authorized user (e.g., the physician) can manually select or update the skill or engagement level for the patient. In addition or alternative to the processed freeform text input from the patient, data collected by one or more patients sensors 770 and/or data collected by the neuromodulation device 750 can be used to determine the treatment phase and the patient's skill or engagement level. In some examples, a patient may be defaulted into a skill or engagement level such as based on the treatment phase. For example, a patient presumably has little knowledge about the device and neuromodulation treatment during the pre-implant trial period, or further within the one-month post-implant period, and can thus be defaulted into the “novice” track during that time. As the patient advances to later phases along the treatment journey, the patient is presumably more familiar with the device functions and treatment options, and thus can be defaulted into a higher skill or engagement level such as an “intermediate” track or an “expert” track.

At 1030, the treatment phase and patient skill or engagement level as determined at step 1020 can be used to query the database of DOT information provided or created at step 1010 to identify therefrom personalized pre-determined DOT information for the patient that corresponds to the treatment phase and the skill or engagement level of the patient. As discussed above with reference to FIGS. 8A-8B, in an example where the database of DOT information is represented by a two-dimensional data array (e.g., depicted in a table format), the personalized pre-determined DOT information is a table entry with the corresponding treatment phase and the skill or engagement level matching respectively the determined treatment phase and the patient skill or engagement level as determined at step 1020.

At 1040, the personalized DOT information, which include device and treatment information releasable to patient and the device programming capability authorized to the patient, can be provided to the patient on a user interface, such as a user interface on a smartphone as described above with reference to FIGS. 9A-9B. In some examples, in addition to the type and/or amount of information releasable to the patient and the device programming capability authorized to the patient, a personalized mode of interaction with the patient can also be determined in accordance with the treatment phase and the skill or engagement level of the patient as determined at step 1020. The personalized mode of interaction can include, for example, timing, a rate, a pattern, or a schedule of presenting the personalized DOT information to the patient. The personalized DOT information can be provided to the patient in accordance with the personalized mode of interaction at 1040. For example, more frequent presentation or interactive question and answer sessions (e.g., an automated chatbot session) may be given to patients with a higher level of engagement with the device and neuromodulation therapies than those with lower engagement levels.

At 1050, the patient may program the neurostimulation device using the authorized device programming capability included in the personalized DOT information as determined at step 1030, which may include adjusting stimulation parameter values, selecting stimulation programs, or changing device setting of the neuromodulation device. A neuromodulation therapy (e.g., SCS for chronic pain control) can be initiated or adjusted in accordance with the patient programming of the neurostimulation device. In various examples, a change in treatment phase or a change in patient skill or engagement level can be detected periodically or in response to a user (e.g., the patient) request. In response to the detected change, a new query of the database can be initiated to determine updated personalized DOT information (including newly customized device and therapy information and newly authorized device programming capability) for the patient corresponding to the new treatment phase or the new patient skill or engagement level. The newly customized device and therapy information can be timely presented to the patient; and the patient may use the newly authorized device programming capability to adjust neurostimulation therapy to meet his or her needs and achieve the treatment goal.

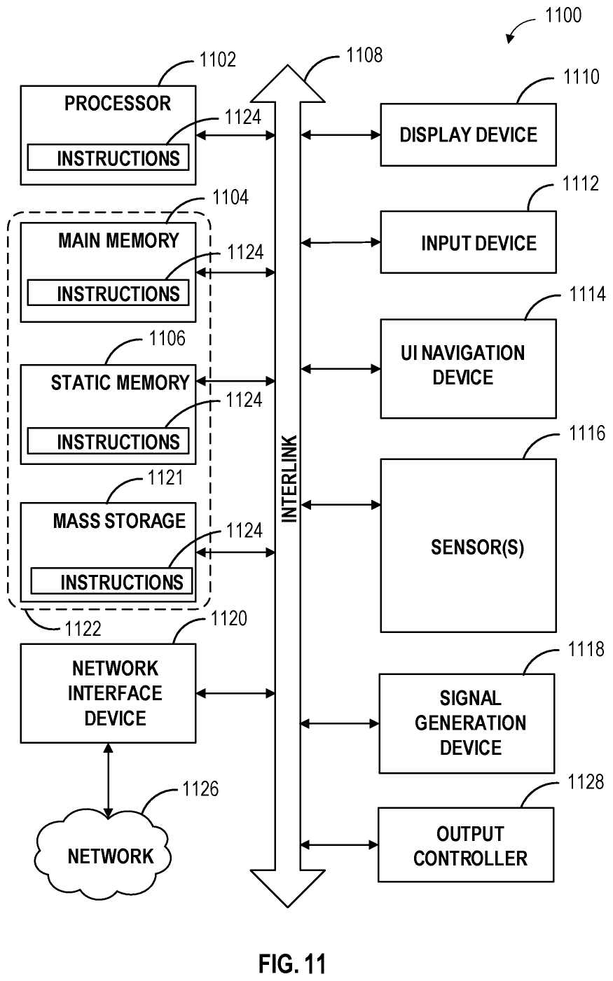

FIG. 11 illustrates generally a block diagram of an example machine 1100 upon which any one or more of the techniques (e.g., methodologies) discussed herein may perform. Portions of this description may apply to the computing framework of various portions of the assisted therapy programming system 700, such as the processor 720 for providing customizable device and therapy information releasable to a patient and the therapy programming capabilities authorized to the patient based on treatment phase and patient skill or engagement level with respect to a neuromodulation device.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 1100 may include a hardware processor 1102 (e.g., a central processing unit (CPU), a graphics processing unit (GPU), a hardware processor core, or any combination thereof), a main memory 1104 and a static memory 1106, some or all of which may communicate with each other via an interlink (e.g., bus) 1108. The machine 1100 may further include a display unit 1110 (e.g., a raster display, vector display, holographic display, etc.), an alphanumeric input device 1112 (e.g., a keyboard), and a user interface (UI) navigation device 1114 (e.g., a mouse). In an example, the display unit 1110, input device 1112 and UI navigation device 1114 may be a touch screen display. The machine 1100 may additionally include a storage device (e.g., drive unit) 1116, a signal generation device 1118 (e.g., a speaker), a network interface device 1120, and one or more sensors 1121, such as a global positioning system (GPS) sensor, compass, accelerometer, or other sensor. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1116 may include a machine readable medium 1122 on which is stored one or more sets of data structures or instructions 1124 (e.g., software) embodying or utilized by any one or more of the techniques or functions described herein. The instructions 1124 may also reside, completely or at least partially, within the main memory 1104, within static memory 1106, or within the hardware processor 1102 during execution thereof by the machine 1100. In an example, one or any combination of the hardware processor 1102, the main memory 1104, the static memory 1106, or the storage device 1116 may constitute machine readable media.

While the machine readable medium 1122 is illustrated as a single medium, the term “machine readable medium” may include a single medium or multiple media (e.g., a centralized or distributed database, and/or associated caches and servers) configured to store the one or more instructions 1124.

The term “machine readable medium” may include any medium that is capable of storing, encoding, or carrying instructions for execution by the machine 1100 and that cause the machine 1100 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1124 may further be transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1100, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for providing customizable interactive healthcare services to a patient in relation to neuromodulation device treatment, the system comprising:
a memory device, configured to store and maintain a database of device operation and treatment (DOT) information of a neuromodulation device, the database of DOT information being categorized according to a plurality of pre-determined treatment phases and a plurality of pre-determined patient skills or engagement levels with the neuromodulation device; and
a processor configured to:
determine a treatment phase and a skill or engagement level of the patient at the determined treatment phase using patient state information;
query the database to identify therefrom personalized DOT information that corresponds to the determined treatment phase and the skill or engagement level of the patient; and
provide the personalized DOT information to the patient on a user interface.

2. The system of claim 1, wherein the processor is further configured to determine a personalized mode of interaction with the patient based on the determined treatment phase and the skill or engagement level of the patient, and to provide the personalized DOT information to the patient in accordance with the personalized mode of interaction.

3. The system of claim 1, wherein the patient state information includes text or voice input from the patient via the user-interface device,
wherein the processor is configured to process the text or voice input using natural language processing, and to determine the treatment phase and the skill or engagement level of the patient using the processed text or voice input.

4. The system of claim 1, wherein the processor is configured to:
detect a change in treatment phase or a change in patient skills or engagement level;
query the database to update the personalized DOT information; and
provide the updated personalized DOT information to the patient on the user interface.

5. The system of claim 1, wherein the plurality of pre-determined treatment phases include a plurality of pre-determined temporal phases representing respective times elapsed from a medical event or a predefined milestone.

6. The system of claim 1, wherein the plurality of pre-determined treatment phases include a plurality of pre-determined stages of patient experience with a neuromodulation therapy.

7. The system of claim 1, wherein the plurality of pre-determined patient skills or engagement levels include a novice level, an intermediate level, and an expert level,
wherein the processor is configured to determine the skill or engagement level of the patient as one of the novice level, the intermediate level, or the expert level using the patient state information.

8. The system of claim 1, wherein the plurality of pre-determined patient skills or engagement levels include a plurality of pre-determined patterns of patient interaction with the neuromodulation device to adjust a neuromodulation therapy parameter,
wherein the processor is configured to determine a device interaction pattern of the patient using the patient state information, and query the database to identify therefrom the personalized DOT information that corresponds to the determined treatment phase and the determined device interaction pattern.

9. The system of claim 1, wherein the patient state information includes patient past interactions with the neuromodulation device,
wherein the processor is configured to track the patient past interactions with the neuromodulation device over time, and to determine the skill or engagement level of the patient based on the tracked patient past interactions.

10. The system of claim 1, wherein the processor is configured to initiate a diagnostic test of patient performance in interacting with the neuromodulation device, and to determine the skill or engagement level of the patient based on the patient performance in the diagnostic test.

11. The system of claim 1, wherein the DOT information in a first category of pre-determined treatment phase or pre-determined patient skill or engagement level is different from the DOT information in a second category of pre-determined treatment phase or pre-determined patient skill or engagement level.

12. The system of claim 1, wherein the database of DOT information includes categorized device and therapy information releasable to the patient,
wherein the pre-determined patient skills or engagement levels include a first skill or engagement level and a second skill or engagement level, the second skill or engagement level representing a lower level of skill or engagement with the neuromodulation device than the first skill or engagement level,
wherein the DOT information corresponding to the first skill or engagement level includes a higher volume or more advanced device and therapy information releasable to the patient than the DOT information corresponding to the second skill or engagement level.

13. The system of claim 1, wherein the database of DOT information includes categorized device programming capability authorized to the patient,
wherein the pre-determined patient skills or engagement levels include a first skill or engagement level and a second skill or engagement level, the second skill or engagement level representing a lower level of skill or engagement with the neuromodulation device than the first skill or engagement level,
wherein the authorized device programming capability corresponding to the first skill or engagement level includes more advanced device programming capability authorized to the patient than the device operation and treatment programming capability corresponding to the second skill or engagement level.

14. A method of providing customizable interactive healthcare services to a patient in relation to neuromodulation device treatment, the method comprising:
providing a database of device operation and treatment (DOT) information of a neuromodulation device, the database of DOT information being categorized according to a plurality of pre-determined treatment phases and a plurality of pre-determined patient skills or engagement levels with the neuromodulation device;
determining a treatment phase and a skill or engagement level of the patient at the determined treatment phase using patient state information;
querying the database to identify therefrom personalized DOT information that corresponds to the determined treatment phase and the skill or engagement level of the patient; and
providing the personalized DOT information to the patient on a user interface.

15. The method of claim 14, comprising:
determining a personalized mode of interaction with the patient based on the determined treatment phase and the skill or engagement level of the patient; and
providing the personalized DOT information in accordance with the personalized mode of interaction.

16. The method of claim 14, wherein the patient state information includes text or voice input from the patient via the user-interface device,
wherein determining the treatment phase and the skill or engagement level of the patient includes processing the text or voice input using natural language processing, and determining the treatment phase and the skill or engagement level of the patient using the processed text or voice input.

17. The method of claim 14, wherein the plurality of pre-determined treatment phases include a plurality of pre-determined temporal phases representing respective times elapsed from a medical event or a predefined milestone, or plurality of pre-determined stages of patient experience with a neuromodulation therapy.

18. The method of claim 14, wherein the DOT information in a first category of pre-determined treatment phase or pre-determined patient skill or engagement level is different from the DOT information in a second category of pre-determined treatment phase or pre-determined patient skill or engagement level.

19. The method of claim 14, wherein the database of DOT information includes categorized device and therapy information releasable to the patient,
wherein the pre-determined patient skills or engagement levels include a first skill or engagement level and a second skill or engagement level, the second skill or engagement level representing a lower level of skill or engagement with the neuromodulation device than the first skill or engagement level,
wherein the DOT information corresponding to the first skill or engagement level includes a higher volume or more advanced device and therapy information releasable to the patient than the DOT information corresponding to the second skill or engagement level.

20. The method of claim 14, wherein the database of DOT information includes categorized device programming capability authorized to the patient,
wherein the pre-determined patient skills or engagement levels include a first skill or engagement level and a second skill or engagement level, the second skill or engagement level representing a lower level of skill or engagement with the neuromodulation device than the first skill or engagement level,
wherein the authorized device programming capability corresponding to the first skill or engagement level includes more advanced device programming capability authorized to the patient than the device operation and treatment programming capability corresponding to the second skill or engagement level.
